(19) United States
(12) Patent Application Publication
(10) Pub. No.: US 2018/0085360 A1
(43) Pub. Date: Mar. 29, 2018

(54) CENTRALLY ACTING ACETYLCHOLINESTERASE INHIBITORS FOR THE PREVENTION AND/OR TREATMENT OF CHEMICALLY INDUCED NEUROPATHIES AND THE SYMPTOMS THEREOF, AND CORRESPONDING COMPOSITIONS, USES, METHODS AND KIT

(71) Applicants: CENTRE HOSPITALIER UNIVERSITAIRE DE CLERMONT FERRAND, Clermont-Ferrand (FR); UNIVERSITE D'AUVERGNE, Clermont-Ferrand (FR)

(72) Inventors: ALAIN ESCHALIER, CHAMALIERES (FR); JEREMY FERRIER, PARAY-SOUS-BRIAILLES (FR); DAVID BALAYSSAC, BOISSEJOUR (FR); FABIEN MARCHAND, CLERMONT-FERRAND (FR)

(21) Appl. No.: 15/829,211

(22) Filed: Dec. 1, 2017

Related U.S. Application Data
(62) Division of application No. 15/127,439, filed on Sep. 20, 2016.

(30) Foreign Application Priority Data
Mar. 20, 2014 (FR) 1452348

(51) Int. Cl. A61K 31/445; A61K 31/555; A61K 9/00

(52) U.S. Cl. CPC A61K 31/445; A61K 31/555; A61K 9/0053

(57) ABSTRACT

The invention relates to a composition for using in the treatment and/or prevention of chemically induced neuropathies, said composition comprising at least one reversible centrally acting acetylcholinesterase inhibitor. The invention also relates to the corresponding methods, uses and kit.

### 1. FIELD OF THE INVENTION

The field of the invention is the prevention and/or treatment of chemically induced neuropathies, in particular the prevention and/or treatment of neuropathies caused by chemotherapy. Symptoms such as pain and other sensitivity issues associated with these neuropathies may be targeted. More specifically, the invention relates to the use of at least one reversible centrally acting acetylcholinesterase inhibitor for the prevention and/or treatment of chemotherapy induced neuropathies; a composition comprising said inhibitor for use in the prevention and/or treatment of chemotherapy induced neuropathies.

### 2. PRIOR ART

Cancer is one of the leading causes of mortality, and its prevalence increases with the age of the population. In particular, colorectal cancer is the third most frequently diagnosed cancer in industrialized countries (source: Institut National du Cancer, updated on Dec. 27, 2012). One of the treatments indicated for advanced colorectal cancer is the association of a platinum salt (oxaliplatin) with 5-fluorouracil and folinic acid. This treatment is known under protocol name FOLFOX4.

Oxaliplatin (cis-[(1R,2R)-1,2-cyclohexanediamine-N,N′] [oxaloto (2)-O,O′] platinum) is a third-generation platinum salt following cisplatin and carboplatin. Marketing authorizations have been granted for this active ingredient to treat colorectal cancer. Studies have also been done relative to the treatment of ovarian and stomach cancer. Cisplatin is still indicated for the treatment of non-small cell lung cancer, certain sarcomas and ovarian cancer. Carboplatin is authorized to treat ovarian, lung, breast, bladder, esophageal, endometrial and ORL cancers.

Oxaliplatin has the advantage of being less nephrotoxic than cisplatin and less hematotoxic than carboplatin. This molecule is not, however, free of adverse effects.

Aside from the fatigue, nausea and digestive problems traditionally caused by chemotherapy, the main factor limiting oxaliplatin is its neurotoxicity, which is higher than that observed with cisplatin- and carboplatin-based chemotherapies. This is in particular characterized by an acute neurotoxicity observed in approximately 90% of treated patients, i.e., a neurotoxicity occurring from several hours to several days after each administration of oxaliplatin.

More specifically, the neurotoxicity of oxaliplatin is reflected, in particular during its acute phase, by a neuropathy characterized by paresthesia and dysesthesia caused by coldness in the hands, throat, mouth and cheeks, swelling (Raymond et al., Ann Oncol, 1998, 9 (10): 1053-1071) as well as mechanical and thermal hypersensitivity (Binder et al., Eur J Cancer. 2007 December; 43(18):2658-63; Attal et al., Pain. 2009 August; 144(3):245-42).

This neurotoxicity is then chronic over the course of the treatment. Clinical studies have shown that about 50% to 70% of patients were developing a persistent and incapacitating peripheral neuropathy, similar to those observed with carboplatin- and/or cisplatin-based chemotherapies. This type of property was reflected by a primarily sensory attack, persistent paresthesia and feelings of swelling between chemotherapy administrations, as well as spontaneous pain. In the most severe cases, patients develop sensory ataxia accompanied by a loss of superficial and deep sensibility affecting even the slightest daily gesture. A recent study (Park et al., Oncologist. 2011; 16(5):708-16) also demonstrated that the neuropathy was becoming persistent in approximately 80% of affected patients, and could progress to establish itself well after the end of treatment using oxaliplatin and/or cisplatin.

Neuropathic pain is a symptom of neuropathy, defined as a secondary pain related to a lesion or a pathological condition affecting the somatosensory system. It is secondary to peripheral lesions (related nerve fibers) or central lesions (nerve centers involved in transmitting and/or regulating pain messages, for example in the thalamus).

Neuropathic pain has various etiologies. It may be caused by a physical lesion, as is the case with an accident, a surgical procedure, or an amputation (phantom limb). It may also be chemically induced, as occurs following alcohol abuse, metal poisoning (arsenic or thallium, for example), exposure to an environmental chemical agent (organophosphates, for example), a medicament, a fungal or bacterial toxin. It may also follow an acquired or hereditary pathological condition (for example, an amyloidosis, acute idiopathic polyneuritis, an HIV infection, a bacterial infection, shingles, herpes, diabetes, autoimmune disease and/or Fabry's disease).

The physiopathological mechanisms responsible for neuropathic pain are currently not well known. Some studies have shed light on the involvement, inter alia, of GABA receivers, K⁺/Cl⁻ transporters, a disruption in the homeostasis of the Cl⁻ and/or the KCC2 co-transporter (Ferrini F et al, Neural Plast, 2013: 429815; Asiedu M N et al., J. Pain, 2014 Jan. 9), the decrease in the serotonin level (Sumpton J E et al, Handb Clin Neurol 2014; 119:513-27), the involvement of the ATP binding site of the P2X receptors (Chataigneau et al., Front Cell Neurosci, 2013 (30) 7-273), variation in the expression of various ion channels in oxaliplatin-induced neuropathy (Descoeur et al, Embo Molecular Medicine, 2011 3(5): 266-278), and/or a malfunction of the hemato-nervous barrier (Lim T K et al, Pain. 2014 May; 155(5):954-67).

Thus, there is a significant heterogeneity in the molecular and cellular mechanisms at the origin of neuropathies. These physiopathological mechanisms are sometimes very different based on the etiology. For example, a differential involvement has been shown of certain second messengers in posttraumatic neuropathies compared to chemically induced neuropathies (Aley and Levine, Neuroscience. 2002; 111(2):389-97). These two types of neuropathies, although sharing common symptoms, must therefore be considered very separate syndromes. This sub-categorization therefore appears crucial and is also verified in terms of pharmacological treatments. Indeed, although certain molecules have received an indication to treat posttraumatic neuropathic pain, none of them have demonstrated substantial efficacy to ease chemically induced neuropathic pain. This is for example the case of amitriptyline and gabapentin, which are nevertheless recommended as first-line treatment for posttraumatic neuropathic pain (Piano et al., Pain practice, 2014) but that have not provided any relief in patients treated with oxaliplatin (Kautio et al., J. of pain and symptom management, 2008; Mitchell et al., Clin. Colorectal Cancer, 2006).

Neuropathy is therefore a generic term encompassing various nonspecific syndromes, just as the term “cancer” designates the abnormal and pathological cell proliferation within healthy tissue without prejudging the cause, symptoms, histology and treatment to be administered to the patient.

It is therefore scientifically incorrect to place all neuropathies (posttraumatic, lesional, chemically induced) on the same level without taking account of their etiology and the biological mechanisms in play. In other words, and as an example, a single symptom such as hypersensitivity to cold cannot reasonably be linked to a single biological mechanism, a single cause and therefore a single treatment.

As previously stated, traumatic neuropathic pain is different from chemically induced neuropathies and pain associated with chemically induced neuropathies.

For example, the majority of antalgics used for neuropathic pain (as opposed to traumatic pain) have little to no effect on chemically induced neuropathies, with the exception of duloxetine (Finnerup et al., Lancet Neurol. 2015 January; S1474-4422(14)70251-0); Hershman et al., J Clin Oncol. 2014 Jun. 20; 32(18):1941-67).

In other words, in the case of animal models, even if shared symptoms are found (sensory disorders), the physiopathology of these traumatic (ligature of the sciatic nerve), metabolic (diabetes) and toxic (antineoplastic) neuropathies is different: expression or non-expression of kinase proteins, expression or non-expression of NMDA receptors, etc. (Aley and Levine, Neuroscience. 2002; 111(2):389-97). Yet it is the mechanism in play that conditions the treatment, and not the symptom.

To illustrate this point through a daily example, it is not possible to link stomach pain to a single cause (colic, intoxication, autoimmune disease, cancer), and therefore to a single treatment (antispasmodic, antibiotic, immunosuppressant, chemotherapy).

Regarding neuropathies (in particular neuropathic pain and associated sensitivity problems) caused by chemotherapy, such as those caused by platinum salts, it appears that these neuropathies are related, directly dependently, to the length of the course of treatment and the administered dose. Consequently, in order to limit or even eliminate the symptoms of neuropathies caused by treatment using platinum salts, a dosing adaptation is currently done; i.e., a decrease in the administered doses of platinum salts, spacing apart of the treatments, or even interruption of treatments. However, this dosing adaptation—although in some cases making it possible to limit or even prevent induced neuropathic pain—compromises the likelihood of remission and survival of the patient.

Aside from platinum salts, other compounds, medicinal or nonmedicinal, cause neuropathies. In particular, certain medicaments used for chemotherapy such as bortezomib, thalidomide and mitotic spindle poisons also cause neuropathies.

Example of nonmedicinal compounds having a neurotoxicity include alcohol, arsenic, thallium, organophosphate compounds, etc.

To date, treatments have endeavored to reduce symptoms one by one without necessarily managing to act further upstream to treat them in an efficacious and targeted manner. It is thus sometimes indicated to prescribe tricyclic antidepressants, gabapentin, pregabalin, psychotherapy, and patient education to manage and live with the pain. This amounts to handpicking medicaments from the pharmacopeia that could soothe the patient, with no certainty or objective criteria.

### 3. AIMS OF THE INVENTION

The invention in particular aims to offset these drawbacks of the prior art and to propose a therapeutic alternative to mere dosing adaptation.

More specifically, the invention aims, at least in one embodiment, to provide a composition making it possible to prevent and/or eliminate, or at least reduce, the symptoms of chemically induced properties such as pain and sensitivity problems.

The invention also aims to provide, in at least one embodiment, for preventing the development of symptoms of neuropathy caused by the administration of the neurotoxic compounds, including medicinal or nonmedicinal neurotoxic compounds, and in particular a neurotoxic antineoplastic compound.

In particular, in at least one embodiment, one aim of the invention is to propose such a composition that makes it possible to eliminate or at least reduce the symptoms of neuropathy caused by the administration of platinum salts.

### 4. BRIEF DESCRIPTION OF THE INVENTION

These aims, as well as others that will appear later, are achieved using a composition for use in the treatment and/or prevention of chemically induced neuropathies and their symptoms, said composition comprising at least one reversible centrally acting acetylcholinesterase inhibitor.

“Chemically induced neuropathy” refers to neuropathies caused by the administration of or exposure to a neurotoxic compound, this compound being able to be:

either a medicinal compound, in particular an antineoplastic compound having a neurotoxicity;

or a nonmedicinal compound such as alcohol, organophosphate compounds, heavy metals (Pb, As, Hg, TI), fungal or bacterial toxins, certain solvents such as toluene, etc.

“Neurotoxic compound” refers to a compound acting as a poison on the nervous system. Neurotoxic compounds traditionally act by disrupting, or even blocking, the sensory impulse.

“Reversible inhibitor” refers to a substance that bonds to its target non-covalently (hydrogen bond, ionic bond, hydrophobic interaction, etc.). Reversible inhibitors do not undergo a chemical reaction upon binding to their target and can therefore be easily eliminated, unlike reversible inhibitors, which form a stable complex with their target on a permanent basis.

“Centrally acting” refers to any compound capable of crossing the blood-brain barrier to exert its effect on the central nervous system (brain and spinal cord).

The inventors have in fact surprisingly discovered that administering reversible centrally acting acetylcholinesterase inhibitors—usually used to treat dementia, such as Alzheimer's disease—made it possible to prevent, lessen, or even treat chemically induced neuropathies and their symptoms, such as sensitivity problems and pain.

As previously mentioned, neuropathy is a pathology affecting at least one nerve. The symptoms of the neuropathy are varied, but generally comprise problems with sensitivity and pain such as allodynia, hyperesthesia, dysesthesia and ataxia. Neuropathic pain is a neurogenic pain. It should, however, be noted that the neuropathy is not necessarily accompanied by pain. Various factors, activating different signaling routes, may cause such a neuropathy. Consequently, the treatments that may be proposed are quite varied, and their respective efficacy varies based on the physiopathological mechanism.

Treating the neuropathy requires understanding the underlying biological mechanism, and more precisely, that implemented by a specific cause. Although ligature models of the sciatic nerve in rats have made it possible to advance the understanding of posttraumatic properties (amputation, for example), this model cannot be transposed to all clinical realities. In particular, this model does not make it possible to shed any light on polyneuropathies caused by a particular neurotoxic antineoplastic compound.

Likewise, document US-2006/0264455-A1 is known, which describes a model of inflammatory pain after a formol injection. Formol causes tissue lesions and inflammatory pain that does not correspond to any clinical situation, other than the injection or ingestion of formal. As explained by the review by M. Barrot pertaining to the different tests and models used to simulate pain in rodents, the formol injection is a model of inflammatory pain and not a model of narcotic pain, including chemically induced neuropathic pain (M. Barrot, Neurosciences 211 (2012) 39-50). Indeed, neuropathic pain is a direct consequence of a lesion or a pathology primarily affecting the nervous system. Conversely, inflammatory pain results from a lesion or an infection. The immune system releases chemical mediators that may damage the nervous system, in a secondary manner. The formol injection model is therefore a short-term inflammatory pain model.

Furthermore, this document indicates that huperzine makes it possible to treat pain by inhibiting the NMDA receptors. However, antagonists of the NMDA receptors are known for their anti-nociceptive properties in the sciatic nerve ligature model (Mao et al., Brain Res. 1993 Mar. 5, 605(1):164-8). These different elements clearly illustrate the complexity of the field and the complete impossibility of reasoning linearly: neuropathy=1 cause=1 mechanism=1 treatment.

Among the whole range of possibilities, the inventors have discovered that reversible centrally acting acetylcholinesterase inhibitors make it possible to prevent, reduce, or even eliminate the symptoms of chemically induced neuropathies, in particular pain and sensitivity problems.

In other words, administering reversible centrally acting acetylcholinesterase inhibitors makes it possible to prevent and/or reduce, or even eliminate, the neurotoxicity of certain neurotoxic compounds, whether medicinal or nonmedicinal, and in particular neurotoxic antineoplastic compounds, both in the chronic phase and the acute phase.

In the case of antineoplastic compounds, the composition according to the invention is of great therapeutic interest. The reversible centrally acting acetylcholinesterase inhibitors indeed make it possible to prevent the neuropathy from becoming chronic over the course of chemotherapy rounds. Consequently, the use of such inhibitors with a preventive and curative effect regarding neuropathic pain (or even other sensitivity disorders) provides considerable comfort to the patient while making it possible to maintain the recommended rhythm of the administrations and the antineoplastic dosing regimens, while avoiding interruptions in patient treatment. Thus, the patient's chances with respect to the disease, after treatment, would be increased.

According to the invention, said reversible centrally acting acetylcholinesterase inhibitor is chosen from the group comprising donepezil, rivastigmine, galantamine, one of the pharmaceutically acceptable salts thereof, one of the pharmaceutically acceptable enantiomers thereof, one of the pharmaceutically acceptable derivatives thereof, or a mixture thereof.

These inhibitors have the advantage of having been approved to treat dementia in particular, in humans. Thus, the proof of their efficacy and relative safety in humans has already been provided.

The IUPAC names of the reversible centrally acting acetylcholinesterase inhibitors suitable for implementation of the invention are as follows:
donepezil (RS)-2-[(1-benzyl-4-piperidyl)methyl]-5,6-dimethoxy-2,3-dihydroindene-1-one);
rivastigmine (S)-3-[1-(dimethylamino)ethyl]phenyl N-ethyl-N-methylcarbamate);
galantamine (4aS,6R,8aS)-5,6,9,10,11,12-hexahydro-3-methoxy-11-methyl 4aH-[1]benzofuro[3a,3,2-ef] [2] benzazepin-6-ol).

“Pharmaceutically acceptable salt” refers to inorganic or organic acid salts such as hydrochlorides, hydrobromides, sulfates, nitrates, phosphates, formates, acetates, oxalates, succinates, maleates, fumarates, saccharates, or non-toxic basic salts such as sodium, potassium, calcium, magnesium, zinc.

Preferably, said inhibitor is chosen from the group comprising donepezil, a pharmaceutically acceptable salt thereof, a pharmaceutically acceptable enantiomer thereof, a pharmaceutically acceptable derivative or mixture thereof; still more preferably, said inhibitor is a donepezil salt, even more preferably, said inhibitor is donepezil hydrochloride. Donepezil causes few side effects, which is appreciable for patient who for example must bear the adverse effects of antineoplastic chemotherapy. Donepezil hydrochloride is currently marketed under its trade name Aricept™ by the Pfizer laboratories (United States).

In particular, the composition according to the invention is used to treat and/or prevent the symptoms of chemically induced neuropathies.

Advantageously, the composition according to the invention is used to treat and/or prevent the symptoms of neuropathies caused by a neurotoxic compound.

Preferably, said neurotoxic compound is an antineoplastic compound chosen from among a platinum salt, bortezomib, thalidomide, a mitotic spindle poison or mixtures thereof, said composition comprising at least one reversible centrally acting acetylcholinesterase inhibitor.

Still more preferably, said antineoplastic compound is a platinum salt chosen from among carboplatin, cisplatin, oxaliplatin and mixtures thereof.

Indeed, the inventors have discovered that administering reversible centrally acting acetylcholinesterase inhibitors in parallel with and/or upstream from chemotherapy using platinum salts makes it possible to prevent and/or treat the development of the associated neuropathy.

Platinum salts have the drawback of causing many side effects that require a dosing adaptation, or even stopping treatment, thereby compromising the patient's chances. The inventors have discovered that administering donepezil, in particular donepezil hydrochloride, made it possible to reduce the signs of neuropathy caused by these neurotoxic compounds.

To date, patients on chemotherapy with a platinum salt and developing a chemically induced neuropathy are treated using conventional treatments, typically used in traumatic neuropathic pain and with very few results (Finnerup et al., Lancet Neurol. 2015; Hershman et al., J Clin Oncol. 2014). And for good reason: this therapeutic approach does not take account of the specificity of the mechanism in play in platinum salt neurotoxicity. The inventors, through their invention, therefore offer an effective therapeutic alternative to patients on chemotherapy using platinum salts, in particular oxaliplatin.

Said antineoplastic compound may, also or alternatively, be chosen from among mitotic spindle poisons. This class of antineoplastics blocks the cell division of cancer cells by disrupting the mitotic spindle, and in particular by blocking the polymerization or depolymerization of the microtubules. The cancer cells are distinguished from noncancerous cells in that they proliferate much more quickly, the frequency of the mitoses therefore being higher than that of noncancerous cells. The use of mitotic spindle poisons therefore makes it possible to more specifically target cancer cells over noncancerous cells.

In particular, the mitotic spindle poison can be chosen from among compounds in the taxane class, compounds in the epothilone class, Madagascar periwinkle alkaloids or mixtures thereof.

Said neurotoxic antineoplastic compound may, also or alternatively, be chosen from among compounds in the epothilone family. Epothilones are a class of antineoplastics acting like the compounds in the taxane family, but will be more efficacious and cause fewer adverse effects. Epothilones fix on the αβ-tubulin heterodimer and thus block the polymerization of the microtubules.

Said neurotoxic antineoplastic compound may, also or alternatively, be chosen from among compounds in the taxane family; preferably from among the group comprising paclitaxel, docetaxel and mixtures thereof. Paclitaxel (Taxor™, Abrasane™) is an antimitotic in particular used to treat breast, ovarian and lung cancer. Docetaxel (Taxotere™) is in particular used to treat breast cancer.

Said compound may, also or alternatively, be chosen from among Madagascar periwinkle alkaloids; preferably from among the group comprising vincristine, vinblastine and mixtures thereof. Vincristine (marketed under the name Oncovin) is an antimitotic in particular used to treat lung cancer, non-Hodgkin's lymphoma and Hodgkin's lymphoma, leukemia and multiple myeloma). Vinblastine (marketed under the names Velban and Alkaban-AQ) is used to treat ovarian, bladder, kidney, breast, testicular cancers and lymphomas.

Said compound may, also or alternatively, be chosen from among bortezomib, thalidomide and mixtures thereof. Bortezomib (Velcade®) is a proteasome inhibitor, in particular indicated to treat multiple myeloma. Thalidomide (Celgene®, Immunoprin®, Thalidomid®) is in particular used to treat multiple myeloma.

In one embodiment, the composition according to the invention comprises between 1 mg and 25 mg, preferably between 6 mg and 12 mg, still more preferably between 5 to 10 mg, of donepezil hydrochloride. It is of note that 10 mg of donepezil hydrochloride corresponds to 9.12 mg of donepezil.

In another embodiment, the composition according to the invention comprises between 1 and 12 mg of rivastigmine.

In another embodiment, the composition according to the invention comprises between 1 mg and 24 mg of galantamine.

The administered dose may vary based on the weight, age and size of the patient as well as the administration method and the galenic form.

The composition according to the invention may be administered by the enteral route (oral route, perlingual route, rectal route) or by the parenteral route (intramuscular injection, intravenous injection or subcutaneous injection).

In one advantageous embodiment, the composition is administered orally. This administration is preferable because it is more practical and simpler for the patient than an injectable solution. The composition may then be in the form of a film-coated tablet, capsule, sugar-coated tablet, effervescent or dispersible tablet, or drinkable solution such as a syrup, solution, suspension.

The composition according to the invention may also be administered in the form of an extended- or delayed-release tablet.

When the composition according to the invention is in injectable form, it may be in the form of a solution, emulsion or suspension.

In one preferred embodiment, the composition according to the invention is administered before the administration of the neurotoxic compounds, in particular before the administration of neurotoxic antineoplastic compounds. This option makes it possible to increase the efficacy of the reversible centrally acting acetylcholinesterase inhibitor by allowing it time to act and preventing the neuropathy and/or the associated neuropathic pain from developing. The inventors have in fact noted that it is more efficacious to prevent the pain from developing and installing itself rather than treating it after the first signs have appeared. The prophylactic administration of at least one reversible centrally acting acetylcholinesterase inhibitor, before the administration of at least one neurotoxic compound, in particular a platinum salt, makes it possible to avoid the development of the neuropathic pain.

The neurotoxic antineoplastic compounds are as defined above.

Advantageously, the composition according to the invention is first administered at a dose of 5 mg/day for a period of 4 to 6 weeks, then a dose of 10 mg/day for a period of 4 to 6 weeks.

It is possible to consider gradually increasing the dose of the composition according to the invention by increasing plateau of 5 mg/day, each plateau being able to last between 2 and 4 weeks, such that the patient gradually becomes accustomed to the composition, until reaching a maximum dose of 10 mg/day, or even 25 mg/day.

In another interesting embodiment, the composition according to the invention is administered at a regular time.

According to the invention, the composition further comprises at least one pharmaceutically acceptable adjuvant. Adjuvant examples are water, preservatives, antioxidants, dyes, solvents, carriers, stabilizers, thickeners, lubricants, perfumes or excipients making it possible to increase the action of the reversible centrally acting acetylcholinesterase inhibitor, its absorption, its metabolism and/or its excretion.

Another aim of the invention relates to the use of at least one reversible centrally acting acetylcholinesterase inhibitor to treat and/or prevent chemically induced neuropathies and their symptoms (in particular neuropathic pain and associated sensitivity disorders).

Such chemically induced neuropathies may be induced by the administration of neurotoxic compound. Said reversible centrally acting acetylcholinesterase inhibitor and said neurotoxic compound are as defined above.

Said reversible centrally acting acetylcholinesterase inhibitor may be chosen from among the group comprising donepezil, rivastigmine, galantamine, a pharmaceutically acceptable salt thereof, a pharmaceutically acceptable enantiomer thereof, a pharmaceutically acceptable derivative thereof or mixtures thereof.

Particularly advantageously, the invention relates to the use of at least one reversible centrally acting acetylcholinesterase inhibitor to prevent and/or treat neuropathies and their symptoms (in particular neuropathic pain and associated sensitivity disorders) caused by a neurotoxic antineoplastic compound chosen from among a platinum salt, a compound from the taxane family, a Madagascar periwinkle alkaloid, bortezomib, thalidomide, or mixtures thereof.

The reversible acetylcholinesterase inhibitors and the neurotoxic antineoplastic compounds are as defined above.

In one interesting embodiment, the invention relates to the use of a donepezil salt, preferably donepezil hypochlorite, to prevent and/or treat the symptoms of neuropathies (in particular neuropathic pain and/or associated sensitivity disorders) caused by the administration of at least one platinum salt.

The platinum salt may be chosen from among carboplatin, cisplatin, oxaliplatin and mixtures thereof.

Another aspect of the invention relates to a method for preventing and/or treating neuropathies and their symptoms (in particular neuropathic pain and associated sensitivity disorders) caused by exposure to or administration of a neurotoxic compound to a patient, comprising a step for exposure to or administration of at least one neurotoxic compound and a step for administering at least one reversible centrally acting acetylcholinesterase inhibitor.

Advantageously, said reversible centrally acting acetylcholinesterase inhibitor is chosen from among the group comprising donepezil, rivastigmine, galantamine, a pharmaceutically acceptable salt thereof, a pharmaceutically acceptable enantiomer thereof, a pharmaceutically acceptable derivative thereof or a mixture thereof.

Preferably, said neurotoxic compound is an antineoplastic compound chosen from among a platinum salt, a mitotic spindle poison, bortezomib, thalidomide or a mixture thereof. The mitotic spindle poisons are as defined above.

Preferably, the reversible centrally acting acetylcholinesterase inhibitor is donepezil, and more preferably, donepezil hypochlorite.

In one particularly advantageous embodiment of the method according to the invention, the step for administering at least one reversible centrally acting acetylcholinesterase inhibitor is done before the step for administering said neurotoxic compound.

This alternative of the method according to the invention is particularly interesting when said neurotoxic compound is an anti-neoplastic compound, as defined above, and in particular when said antineoplastic compound is a platinum salt. The inventors have in fact shown that administering a reversible centrally acting acetylcholinesterase inhibitor before the round of chemotherapy comprising at least one platinum salt makes it possible to avoid the development of the symptoms of neuropathy (in particular pain and the associated sensitivity disorders), or at the very least, to delay or lessen its intensity.

Preferably, said reversible centrally acting acetylcholinesterase inhibitor is administered to the patient at a regular time.

In one interesting embodiment of the method according to the invention, the step for administering said reversible centrally acting acetylcholinesterase inhibitor is done by successive plateaus of 4 to 6 weeks in order to increase the dose by 5 mg per plateau. In other words, the step for administering said reversible acetylcholinesterase inhibitor comprises:

a first sub-step lasting 2 to 4 weeks, during which the patient receives a dose of 5 mg/day; and

a second sub-step during which the patient receives a dose of 10 mg/day.

Preferably, the second sub-step lasts between 4 and 6 weeks.

The steps for administering said reversible centrally acting acetylcholinesterase inhibitor may comprise at least two sub-steps each lasting 4 to 6 weeks, the daily dose of at least one reversible acetylcholinesterase inhibitor being kept constant for the duration of each plateau and being increased by 5 mg/day at the end of each of the plateaus.

Another aim of the invention relates to a kit comprising at least one neurotoxic antineoplastic compound chosen from the group comprising a platinum salt, bortezomib, thalidomide, a mitotic spindle poison or a mixture thereof; and a reversible centrally acting acetylcholinesterase inhibitor. The mitotic spindle poisons are as defined above.

Said reversible centrally acting acetylcholinesterase inhibitor and said antineoplastic compound are as defined above.

### 5. LIST OF FIGURES

Other features and advantages of the invention will appear more clearly upon reading the following description of one preferred embodiment, given as a simple illustrative and non-limiting example, and the appended drawings, in which:

FIG. 1 shows a graph illustrating the effect of an administration of donepezil (5 mg/kg, administered orally) on mechanical allodynia following a chronic administration of oxaliplatin in rats;

FIG. 2 shows a graph illustrating the results of an administration of donepezil (5 mg/kg, administered orally) on cold allodynia following chronic oxaliplatin administration in rats;

FIG. 3 shows a graph illustrating the preventive effect of the chronic administration of donepezil (5 mg/kg, 2 times per day, administered orally) on mechanical allodynia following chronic oxaliplatin administration in rats;

FIG. 4 shows a graph demonstrating the preventive effect of chronic donepezil administration (5 mg/kg, 2 times per day, administered orally) on cold allodynia following chronic oxaliplatin administration;

FIG. 5 shows a graph illustrating the effect of a donepezil administration (5 mg/kg, administered orally) on anxiety in rats treated or not treated with oxaliplatin.

### 6. DESCRIPTION OF ONE EMBODIMENT OF THE INVENTION

The general principle of the invention lies in the administration of at least one reversible centrally acting acetylcholinesterase inhibitor to prevent and/or treat chemically induced neuropathies and their symptoms.

In particular, the invention comprises, in one of its particularly interesting aspects, the administration of at least one reversible centrally acting acetylcholinesterase inhibitor upstream from and/or in parallel with the administration of a neurotoxic antineoplastic compound chosen from among a platinum salt, bortezomib, thalidomide, a mitotic spindle poison such as a compound from the taxane family or a compound from the epothilone family, a Madagascar periwinkle alkaloid, or a mixture thereof.

Platinum salts, such as oxaliplatin, carboplatin and cisplatin, are currently used to treat certain forms of cancer. They all have a more or less significant neurotoxicity based on the molecules, oxaliplatin being the most neurotoxic of the platinum salts. This neurotoxicity is acutely reflected following each administration of oxaliplatin to the patient, and becomes chronic as the patient continues to receive doses of platinum salts (cisplatin, carboplatin and oxaliplatin). This neurotoxicity is reflected by the appearance of signs encountered in neuropathic pain such as burning sensations, tingling, swelling, allodynia, paresthesia, which persist well after the treatment has been stopped. The inventors have surprisingly observed that administering a daily dose of a reversible centrally acting acetylcholinesterase inhibitor in parallel with an injection of platinum salts made it possible to reduce the neuropathic pain caused by these salts, and to prevent the development of chronic pain. They also noted that administering a reversible centrally acting acetylcholinesterase inhibitor before administering a platinum salt made it possible to further reduce the neuropathic pain, or even to prevent it. The advantages of the invention are demonstrated by the following comparative tests.

### 6.1. Experimental Protocol

6.1.1. Experimental Conditions

As previously indicated, oxaliplatin is a third-generation platinum salt that follows carboplatin and cisplatin. Oxaliplatin being the most neurotoxic of these salts, the experiments were conducted using oxaliplatin on male Sprague-Dawley rats.

Oxaliplatin (Leancare, United Kingdom) was dissolved in a 5% glucose solution at the final concentration of 2 mg/ml in order to obtain an injectable volume of 1 ml/kg. The oxaliplatin solution is injected 2 times per week to animals intravenously for a period of 4.5 weeks (cumulative dose 18 mg/kg). The animals in the control group received an injection of 5% glucose by the same route, at the same frequency and the same volume.

Donepezil hydrochloride is used as an example reversible centrally acting acetylcholinesterase inhibitor (Aricept™ 10 mg, Pfizer, United States). To simplify the explanation, donepezil hydrochloride will be referred to in the examples below as “donepezil”. It assumes the form of film-coated tablets. These tablets were reduced to a fine powder, and placed in suspension in water containing 0.5% carboxymethylcellulose (Sigma Aldrich, France) in order to obtain a solution at 4 mg/ml. The donepezil was administered orally at the dose of 5 mg/kg. The animals in the control group simply received an administration of 0.5% carboxymethylcellulose by the same route, at the same volume and the same frequency.

Different groups of animals were created in order to compare the conditions:

a control group of 8 rats only receiving the control solutions (5% glucose and 0.5% carboxymethylcellulose) (Sham group);

a group of 8 rats having received an oxaliplatin solution and a 0.5% carboxymethylcellulose solution (group O);

a group of 8 rats having received a 5% glucose solution and a donepezil solution (group D);

a group of 8 rats having received an oxaliplatin solution and a donepezil solution (group O-D).

6.1.2. Behavioral Tests

Allodynia, cold or mechanical, corresponds to a sensation of pain felt following a stimulus that is normally painless in a healthy individual. This phenomenon is characteristic of neuropathic pain. Different standardized behavioral tests were conducted to assess the neuropathic pain in the animals.

Von Frey Test: Mechanical Allodynia

The Von Frey electronic apparatus (Chaplan et al., 1994) includes a portable sleeve made up of an elastic tip connected to a force sensor. The rats are placed in Plexiglas boxes, arranged on a raised grate. After a habituation period of the rats, the tip is applied perpendicularly to the center of the 5 plantar pads of each of rear paw. The applied pressure is gradually increased until it causes a withdrawal reflects of the paw. The intensity of the stimulus, the maximum applied pressure (expressed in grams) is recorded by the device upon withdrawal of the paw.

Social Interaction Test: Anxiety

This test takes place in an arena equipped with a camera in which two animals are placed in one another's presence for 10 minutes. The purpose of this test is to measure the interaction time between the animals. The two studied animals are always part of the same experimental condition group to prevent the behavior of one rat from influencing the other. The rats interact with one another by sniffing, grooming, chasing, etc. An increase in interactions indicates a non-anxious state of the rat, while a decrease and tendency toward prostration indicates an anxious state of the animal. At the beginning of the experiment, the animals are placed in opposite corners of the arena and the handler leaves the room. The arena is cleaned between two experiments to prevent the odors from the previous rats from disrupting the results.

Tail Immersion Test: Cold Allodynia

The rats' tails are submerged in a cold water bath (10° C.). The immersion is maintained until the animal withdraws its tail. The latency of the withdrawal is clocked. Each measurement is done in triplicate.

### 6.2. Results

The results were analyzed using the repeated measurement ANOVA method (2 factors).

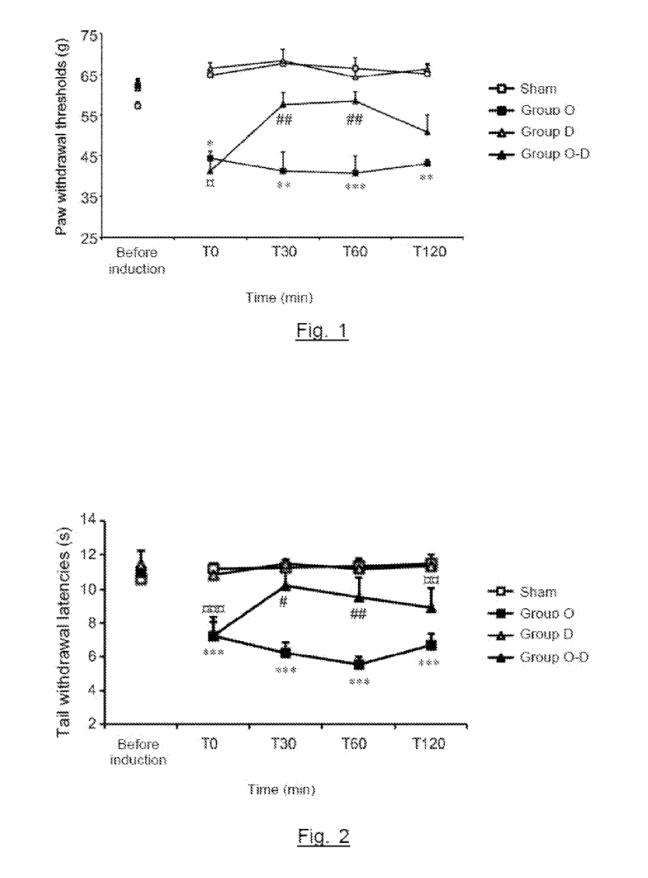

In FIGS. 1 to 6, the stars indicate the significance of the results between group O and the Sham group according to the following scale:

*=p<0.05;

**=p<0.01;

***=p<0.001.

The circles indicate the significance of the results between group O and group D using the following scale:

¤=p<0.05;

¤ ¤=p<0.01;

¤ ¤ ¤=p<0.001.

The pound signs indicate the significance of the results between group O and group O-D according to the following scale:

#=p<0.05;

##=p<0.01;

###=p<0.001.

6.2.1. Effect of a Dose of Donepezil on Oxaliplatin-Induced Neuropathy

The effects of the administration of the 5 mg/kg dose of donepezil on static mechanical and cold allodynia were studied. For these experiments, the donepezil was administered to the rats at the end of the oxaliplatin injection protocol, i.e., 31 days after the first administration of oxaliplatin, in order to assess the curative effect of donepezil. The trials were conducted over a period of 3 hours, on all 4 groups of rats. The time t0 corresponds to the moment of the injection. The results are shown in FIGS. 1 and 2.

FIG. 1 shows the results of the experiment on static allodynia, assessed using the electronic Von Frey test. As one can see, the animals treated with oxaliplatin (group O) showed a decrease in the paw withdrawal threshold relative to the Sham group. In other words, the animals in group O feel pain at a lower stimulation compared to the Sham group. This is consistent with the clinical data collected in humans. Conversely, one notes that the animals in group O-D show a significant improvement in the paw withdrawal threshold after 30 min., which lasts up to 60 min. after the administration of donepezil.

FIG. 2 shows the results of the experiment on cold allodynia, assessed using the tail immersion test in 10° C. water. The observations are similar to those of FIG. 1. Group O has a significantly lower tail withdrawal threshold than the Sham group, which means that the rats treated with oxaliplatin alone have an increased sensitivity to cold compared to the rats treated by the vehicles. The results of the O-D group demonstrate that administering donepezil allows the rats to increase their tolerance for cold, or in other words, to decrease cold allodynia, after 30 minutes and up to 120 minutes after the end of the administration.

In conclusion, these tests demonstrate that administering a reversible centrally acting acetylcholinesterase inhibitor makes it possible to considerably reduce the symptoms of neuropathic pain caused by exposure to platinum salts. The reversible centrally acting acetylcholinesterase inhibitor makes it possible to reverse the signs of neuropathic pain in just 30 minutes. These results demonstrate that administering a reversible centrally acting acetylcholinesterase inhibitor makes it possible to eliminate the neuropathic pain caused by oxaliplatin once that pain has set in.

6.2.2. Preventive Effect of Repeated Administration of Donepezil on Oxaliplatin-Induced Neuropathy

The experiment in point 6.2.2 was conducted on the same animal model reproducing the oxaliplatin-induced neuropathy, with the exception that the study was conducted between D0 and D28. The rats received a dose of 5 mg/kg of donepezil twice per day for 2 weeks (from day 14 to day 28). The treatment with donepezil was established before the tactile and thermal allodynia symptoms appeared in order to assess its preventive effect.

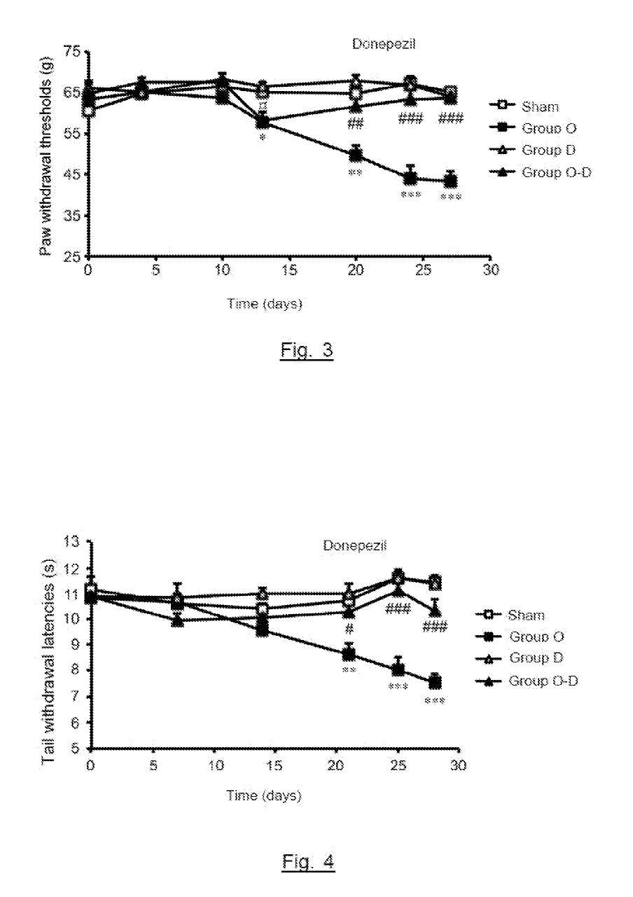

FIG. 3 shows the results on the study of mechanical allodynia, evaluated using the Von Frey test. FIG. 4 shows the results of the effect of a chronic administration of donepezil on cold allodynia, evaluated using the tail immersion test.

As can be seen in FIGS. 3 and 4, group O had an increased sensitivity to mechanical stimulation (FIG. 3) and cold stimulation (FIG. 4) compared to the Sham group. This observation is consistent with the results of the studies of experiment 6.2.1 and clinical data in humans.

Conversely, FIGS. 3 and 4 demonstrate that the animals treated chronically with donepezil (group O-D) have a significantly higher pain threshold than animals treated using oxaliplatin alone (group O). Interesting fact: the animals in group O-D have similar cold and mechanical response thresholds compared to the Sham and D groups. This demonstrates that a chronic administration of a reversible centrally acting acetylcholinesterase inhibitor makes it possible to prevent the development of platinum salt-induced neuropathic pain, until the end of the study.

6.2.3. Effect of an Administration of Donepezil on Oxaliplatin-Induced Neuropathy: Anxiety Evaluation

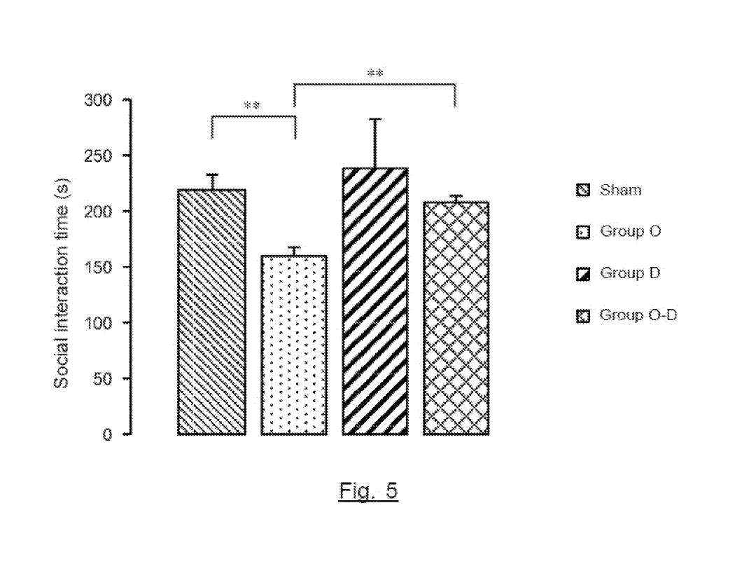

The effect of donepezil on anxiety was studied in all four groups using the social interaction test. The test was done at the end of the oxaliplatin administration protocol, i.e., on day 31. A dose of 5 mg/kg was administered to the animals in groups D and O-D 30 minutes before the test. The results of the social interaction test are shown in FIG. 5. The shorter the social interaction time is, the more anxious the animal is.

As one can see, the animals in group O are significantly more anxious than the animals in the Sham group. The animals in group D have an anxiety level similar to those of the Sham group, the difference not being significant. However, the animals in group O-D have an anxiety level equivalent to that of the Sham group and significantly lower than that of group O. In other words, the administration of a centrally acting acetylcholinesterase inhibitor makes it possible to reduce the anxiety associated with neuropathic pain caused by exposure to platinum salts.

### 7. CONCLUSION

As has been demonstrated, platinum salts, and oxaliplatin in particular, generally cause neuropathic pain in the treated animals, which is in particular manifested by a mechanical allodynia, a cold allodynia; it is accompanied by a significantly higher anxiety level than in non-treated subjects. Administering at least one reversible centrally acting acetylcholinesterase inhibitor after administering at least one platinum salt makes it possible to reduce the symptoms of neuropathic pain, as was demonstrated in point 6.2.1. Such an administration, done chronically, also makes it possible to prevent the development of neuropathic pain, as was proven in point 6.2.2. Lastly, administering at least one reversible centrally acting acetylcholinesterase inhibitor also makes it possible to reduce the anxiety of the patient suffering from neuropathic pain.

Consequently, the composition according to the invention makes it possible to reduce and prevent sensory manifestations, and in particular pain, related to platinum salt-induced neuropathies. This is interesting because rounds of chemotherapy using platinum salt are planned. It is therefore possible to administer at least one reversible acetylcholinesterase inhibitor preventively, before or during the round of chemotherapy using platinum salts, in order to prevent the development of neuropathic pain.

The inventors therefore propose an effective and targeted therapeutic alternative to the current treatments focused on attenuating symptoms.

1. A method for treating a chemically induced neuropathy in a subject, comprising administering to the subject a composition comprising donepezil, or a pharmaceutically acceptable salt thereof, a pharmaceutically acceptable enantiomer thereof, wherein the chemically induced neuropathy is induced by an antineoplastic compound chosen from among a platinum salt, bortezomib, thalidomide, a mitotic spindle poison or a mixture thereof.

2. The method of claim 1, wherein the composition comprises donepezil hydrochloride.

3. The method of claim 1, wherein said antineoplastic compound is a platinum salt chosen from among the platinum salts; preferably from among the group comprising carboplatin, cisplatin, oxaliplatin and mixtures thereof.

4. The method of claim 1, wherein said antineoplastic compound is a mitotic spindle poison chosen from among Madagascar periwinkle alkaloids, compounds in the epothilone class, compounds in the taxane class, or mixtures thereof.

5. The method of claim 4, wherein said neurotoxic antineoplastic compound is chosen from among Madagascar periwinkle alkaloids; preferably from among vincristine, or mixtures thereof.

6. The method of claim 1, wherein said neurotoxic antineoplastic compound is chosen from among bortezomib, thalidomide and mixtures thereof.

7. The method of claim 2, comprising between 1 and 25 mg, preferably between 6 mg and 12 mg, still more preferably between 5 to 10 mg, of donepezil hydrochloride.

8. The method of claim 1, characterized in that the composition is administered orally.

9. (canceled)

10. The method of claim 2, wherein the composition comprises between 6 mg and 12 mg of donepezil hydrochloride.

11. The method of claim 2, wherein the composition comprises between 5 to 10 mg of donepezil hydrochloride.
